SEQUENCE LISTING (2 sequences)

SEQ ID NO: 1 - DNA, 61 bp, Artificial Sequence
  Primer
ggccagtgaattgtaatacgactcactatagggaggcggcttgggtggagtcacatttctc

SEQ ID NO: 2 - DNA, 64 bp, Artificial Sequence
  Primer
ggccagtgaattgtaatacgactcactatagggaggcgggaggagacggtgactgaggttcctg

(19) United States
(12) Patent Application Publication
(10) Pub. No.: US 2007/0042349 A1
(43) Pub. Date: Feb. 22, 2007

(54) Methods for assessing biologic diversity

(76) Inventors: Brenda M. Ogle, Rochester, MN (US); Jeffrey L. Platt, Rochester, MN (US); Marilia I. Cascalho, Rochester, MN (US)

Correspondence Address: FISH & RICHARDSON P.C., PO BOX 1022, MINNEAPOLIS, MN, 55440-1022, US

(21) Appl. No.: 10/554,122

(22) PCT Filed: Apr. 20, 2004
(86) PCT No.: PCT/US04/12058

§ 371 Date: Sep. 11, 2006

Related U.S. Application Data
(60) Provisional application No. 60/464,981, filed on Apr. 24, 2003.

(51) Int. Cl. C12Q 1/70; C12Q 1/68; C12M 3/00

(52) U.S. Cl. 435/5; 435/6; 435/287.2

(57) ABSTRACT

Methods for determining biologic diversity (e.g., lymphocyte receptor diversity or diversity of viral quasispecies) in a subject are described, as well as methods for monitoring a disease in a subject.

### TECHNICAL FIELD

This invention relates to methods of assessing biologic diversity, and more particularly to using random nucleic acid molecules for assessing biologic diversity.

### BACKGROUND

The ability to mount an immune defense against infectious microorganisms and their products, tumors and other environmental challenges, is believed to be a direct function of lymphocyte diversity. See Silins et al., Blood, 98:3739-44 (2001); and Clemente et al., Lab. Invest. 78:619-27 (1998). While the total number of lymphocytes in the blood can be measured with precision, the diversity of the T cell compartment, on which immunocompetence is based, cannot.

In the absence of direct measures of lymphocyte diversity, various indirect methods for estimating diversity have been used. For example, antibodies against variable (V)-region families have been used to characterize lymphocyte populations by flow cytometric analysis. Sheehan et al., Embo J. 8:2313-20 (1989); Langerak et al., Blood 98:165-73 (2001). This approach detects “constant” antigenic determinants shared by many lymphocyte receptor clones and diversity is inferred from the result. As another example, nucleic acids encoding lymphocyte receptors can be amplified by polymerase chain reaction (PCR) using constant region (C) and V family specific primers. Murata et al., Arthritis Rheum. 46:2141-7 (2002). Like FACS analysis, this approach does not differentiate between individual clones of the same family and may fail to detect balanced narrowing (or expansion) of the repertoire.

Diversity can also be estimated by spectratyping or immunoscope. See Pannetier et al., Proc. Natl. Acad. Sci. USA 90:4319-23 (1993); Pannetier et al., Immunol. Today 16:176-81 (1995); and Delassus et al., J. Immunol. Methods 184:219-29 (1995). After V specific families are amplified by PCR, a fluorescently labeled junctional region (J) primer is used for a “run off” PCR reaction, the products of which can be separated on sequencing gels. Amplified lymphocyte receptor families (specified by the primers used in the initial PCR) migrate in a series of bands, each of which corresponds to a different length of the complementarity determining region 3 (CDR3—T cell receptor (TCR) region believed to harbor the largest portion of genetic variability). In normal lymphocyte populations, the CDR3 size distribution is Gaussian for each variable region family and so any alteration in distribution and/or band intensity is attributed to a perturbation of diversity. Unfortunately, V and J combinations cannot be analyzed routinely because over 4,684 V-J family combinations for human T cell receptors exist. Hence, only a small fraction of V-J combinations are analyzed, the choice of which is random and therefore may or may not represent the entire receptor population. In addition, spectratyping does not detect individual clones that may share the same V-J combination and the same CDR3 length.

Still another method of measuring lymphocyte diversity is based on the tenets of limiting dilution analysis and detects the frequency of a given TCR clone. Wagner et al., Pro. Natl. Acad. Sci. USA 95:14447-52 (1998). This method is laborious and is based on the assumption that the frequency of the selected clone represents the frequency of all clones. Thus, a method that can directly and rapidly assess lymphocyte receptor diversity is needed.

### SUMMARY

The invention is based on methods for estimating biologic diversity using random nucleic acid molecules. For example, methods described herein can be used to identify and/or quantify heterogeneous populations of viruses that are contained within an individual (i.e., viral quasispecies). Methods described herein also can be used to probe directly the entire population of lymphocyte receptors. The repertoire of lymphocyte receptor genes is established by rearrangement of germline DNA, resulting in >1000-fold more diversity than the entire genome, and varies between genetically identical individuals. Methods of the invention include hybridizing labeled nucleic acid molecules from the biologic population to be assessed (e.g., viruses or lymphocyte receptors), with a population of random nucleic acid molecules. Diversity is assessed based on the hybridization of the two populations of nucleic acid molecules. As described herein, the frequency of hybridization of the labeled nucleic acids to the random nucleic acid molecules varies in direct proportion to diversity. Methods of the invention can be used clinically to diagnose immunodeficiency stemming from compression of lymphocyte repertoires or to monitor immune reconstitution following hematopoietic cell transplantation. In addition, viral quasispecies can be identified and quantified to guide therapeutic choices and make prognostic assessments.

In one aspect, the invention features a method for determining lymphocyte diversity in a subject. The method includes providing labeled nucleic acid molecules (e.g., RNA or DNA) from a population of the subject's lymphocytes (e.g., T or B lymphocytes), wherein each labeled nucleic acid molecule encodes a lymphocyte receptor or a portion thereof; hybridizing the labeled nucleic acid molecules or fragments of the labeled nucleic acid molecules with a population of random nucleic acid molecules; and determining lymphocyte diversity of the subject by assessing hybridization of the labeled nucleic acid molecules with the population of random nucleic acid molecules. The random nucleic acid molecules within the population can be attached to a solid substrate (e.g., a multiwell plate or membrane, a glass slide, a chip, or a bead). For example, the random nucleic acid molecules can be attached to a bead and hybridization can be assessed by flow cytometry. The solid substrate can include a plurality of discrete regions, wherein each of the discrete regions includes a different random nucleic acid molecule. The labeled nucleic acid molecules can be labeled with a fluorochrome (e.g., fluorescein isothiocyanate (FITC), phycoerythrin (PE), allophycocyanin (APC), or peridinin chlorophyll protein (PerCP)), biotin, or an enzyme. Each labeled nucleic acid molecules can encode a variable region from a T cell receptor (e.g., a complementarity determining region (CDR) 3β chain polypeptide) or a variable portion from a heavy chain or a light chain.

The invention also features a method for monitoring a disease in a subject. The method includes providing labeled nucleic acid molecules from a population of the subject's lymphocytes, wherein each labeled nucleic acid molecule encodes a lymphocyte receptor or a portion thereof; hybridizing the labeled nucleic acid molecules or fragments of the labeled nucleic acid molecules with a population of random nucleic acid molecules; determining lymphocyte diversity of the subject by assessing hybridization of the labeled nucleic acid molecules with the population of random nucleic acid molecules; and comparing the subject's lymphocyte diversity with lymphocyte diversity of a control population, wherein an alteration in the subject's lymphocyte diversity relative to that of the control population indicates a change in the disease. The random nucleic acid molecules can be attached to a solid substrate and labeled as described above. An increase in the subject's lymphocyte diversity can indicate a positive change in the disease. A decrease in the subject's lymphocyte diversity can indicate a negative change in the disease. The disease can be an autoimmune disorder (rheumatoid arthritis or multiple sclerosis), colitis, or a lymphoid disease (e.g., leukemia or lymphoma).

In another aspect, the invention features a method for determining viral diversity in a subject. The method includes providing labeled nucleic acid molecules from a biological sample of the subject, wherein the labeled nucleic acid molecules encode a viral polypeptide (e.g., hemaglutinin, Env, gp120, E1, or E2, or a variable portion thereof); hybridizing the labeled nucleic acid molecules or fragments of the labeled nucleic acid molecules with a population of random nucleic acid molecules; and determining viral diversity of the subject by assessing hybridization of the labeled nucleic acid molecules with the population of random nucleic acid molecules. The random nucleic acid molecules can be attached to a solid substrate and labeled as described above.

The invention also features an article of manufacture that includes a solid substrate, wherein the solid substrate includes random nucleic acid molecules immobilized thereto; and a primer for producing nucleic acid molecules encoding a lymphocyte receptor or a portion thereof or a primer for producing nucleic acid molecules encoding a viral polypeptide. The solid substrate can be a multiwell plate or membrane, a glass slide, a chip, or a bead.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIGS. 1A and 1B are graphs depicting the relationship between the number of hits (as defined as the number of gene chip sites undergoing hybridization) and the number of variants. As indicated in FIG. 1A, the number of hits increases with the number of variants, indicating that the human gene chip can be used to detect random oligonucleotides. In FIG. 1B, the natural log of both axes yielded a linear relationship between hits and variants.

FIG. 2 is a graph depicting the reproducibility of the method for analysis of receptor diversity. Samples from FIG. 1 were studied in three separate experiments to test reproducibility. The slopes of the standard curves were the same statistically; the y intercept varied from experiment to experiment.

FIG. 3 is a graph depicting the relationship between the number of hits and the number of variants for B cells from mice with known variation in B cell diversity using the gene chip method. Splenocytes were harvested from 3-4 week old JH−/−, MBT, QM and WT mice and mononuclear cells were isolated on Ficoll-paque gradients. Total RNA was isolated from the leukocytes and first strand cDNA was generated using a primer designed to bind the constant region of the mouse heavy chain J region plus the T7 polymerase promoter. The custom primer promoted amplification of heavy chain-specific RNA only. Equal amounts of the in vitro transcription product (cRNA) from each mouse and standards (-●-) were hybridized to gene chips and then the chips were stained and analyzed as described in FIG. 1. WT diversity (-▴-) was more than two-fold higher than QM (-▪-) diversity. MBT (-●-) diversity was less than 1 logarithmic unit. Background hybridization was established using JH −/− RNA (-⋄-).

FIG. 4A is a graph depicting B cell heavy chain diversity in mutant mice before and after immunization with KLH. Pre-immunization, WT diversity (●) was more than 2-fold higher than QM (◯) diversity. Post-immunization, WT diversity (●) decreased (4.0×10⁴ different B cell heavy chain clones) while QM (◯) diversity increased (7.5×10³). Background hybridization was established using JH −/− RNA (▪).

FIG. 4B is a graph depicting immune responsiveness to KLH. An ELISA was used to detect levels of anti-KLH antibodies in the serum following immunization. The QM anti-KLH antibody titer was −40% of the wild-type following immunization *P<0.05.

FIG. 5 is a graph depicting the analysis of human T cell diversity using gene chips. Two normal individuals ((-●-), (-◯-)), two thymectomized individuals ((-▪-), (-□-)) and one individual with inflammatory bowel disease (IBD) (-▴-) were analyzed. Background hybridization was established using Jurkat cell hybridization.

FIG. 6 is a graph depicting mean fluorescence as a function of sample diversity.

FIG. 7 is a graph depicting JH4 B cell heavy chain diversity of C57 (wild type) and MBT mice.

### DETAILED DESCRIPTION

In general, the invention provides a method for the direct measurement of biologic diversity (e.g., lymphocyte diversity or viral quasispecies). As used herein, “viral quasispecies” refers to different, but closely related viral variants, within an individual. The method typically employs nucleic acid molecules from the biologic population to be assessed, wherein the nucleic acid molecules encode polypeptides containing one or more variable portions. As used herein, the term “polypeptide” refers to a chain of amino acids, regardless of length or post-translational modification. For example, to assess lymphocyte receptor diversity, each nucleic acid molecule can encode a T cell receptor (TCR) or a B cell receptor (BCR), or a variable portion thereof. To assess viral quasispecies, the nucleic acid can encode a viral polypeptide (e.g., a full-length surface polypeptide or a variable portion thereof). Such nucleic acid molecules are labeled then hybridized with a population of random nucleic acid molecules. Diversity is assessed by the hybridization of the two populations of nucleic acid molecules.

Methods of the invention can be used to estimate diversity of the entire viral quasispecies or lymphocyte repertoire (i.e., all gene segment combinations) at once and is equally capable of measuring B cell and T cell diversity. Furthermore, the methods can be used to estimate diversity of a particular population of cells or immunoglobulins (e.g., IgG or IgM molecules). The method is sufficiently simple and effective to allow widespread application, including the monitoring of immunological disease in subjects, monitoring immune reconstitution following hematopoietic cell transplantation, determining suitable therapies for treatment of a viral infection, and determining prognosis.

Nucleic Acid Molecules from Lymphocytes

To assess lymphocyte receptor diversity, nucleic acid molecules encoding polypeptides containing one or more variable portions are used. Such nucleic acid molecules each can encode an α, β, γ, or δ chain of a TCR, or one or more variable portions from the α, β, γ, or δ chain of a TCR. For example, a variable portion from an a chain of a TCR can be encoded, for example, by one or more of the V_{α}n or J_{α}n variable gene segments. As used herein, “gene segment” refers to a nucleic acid molecule encoding a variable (V), diversity (D), junctional (J), or other region of a TCR or BCR. Gene segments typically are separated from one another in the genome by large stretches of DNA that are not transcribed. Gene segments are composed of coding sequences (exons) that are separated by introns. In some embodiments, the variable portion of the a chain is a hypervariable region (e.g., a complementarity determining region (CDR)) or a portion of a hypervariable region. Thus, in some embodiments, a nucleic acid molecule can encode a CDR, e.g., CDR1, 2, and/or 3, of an α chain or a portion of a CDR. CDR1 and CDR2 of the a chain are encoded by V gene segments; CDR3 is encoded by V and J gene segments.

A variable portion from a β chain of a TCR can be encoded, for example, by one or more of the V_{β}n or J_{β}n gene segments. In some embodiments, the variable portion of a β chain is a hypervariable region (e.g., a CDR) or a portion thereof. Thus, in some embodiments, a nucleic acid molecule can encode a CDR, e.g., CDR1, 2, and/or 3, of a β chain, or a portion of CDR. In the β chain, CDR1 and CDR2 are encoded by V gene segments; CDR3 is encoded by V, D, and J gene segments.

Suitable nucleic acid molecules also can encode a BCR or one or more variable portions of a BCR (e.g., a variable portion of a heavy or light chain of an immunoglobulin, including IgG, IgM, IgD, IgA, and IgE molecules). For example, a variable portion of a heavy chain can be encoded by one of the V_{H}, D, or JH gene segments. In some embodiments, the variable portion is a hypervariable region (e.g., CDR1, 2, and/or 3) or a portion of a hypervariable region. In a light chain (e.g., a κ or λ chain), the variable portion can be encoded by any one of the V_{κ}n or V_{λ}n gene segments, or any one of the J, L, or JL gene segments. In some embodiments, the nucleic acid encodes a hypervariable region (e.g., CDR1, 2, and/or 3) of a light chain or a portion of a hypervariable region.

Populations of nucleic acid molecules encoding a TCR or BCR, or a variable portion of a TCR or BCR, can be isolated from mononuclear cells. In general, a population of mononuclear cells can be obtained from a biological sample then nucleic acids can be extracted from the mononuclear cells. For example, blood (e.g., peripheral blood) or a tissue sample (e.g., biopsy) can be obtained from a subject (e.g., a human) and mononuclear cells isolated from such samples using known techniques. For example, density gradient separation medium (e.g., Ficoll-Paque (Amersham Biosciences, Piscatanaway, N.J.)) can be used to isolate mononuclear cells. Alternatively, negative or positive selection strategies can be used to obtain particular populations of lymphocytes (e.g., T cells or B cells).

Nucleic acids can be obtained from the cells using known techniques. As used herein, “nucleic acid” refers to both RNA and DNA, including total RNA and genomic DNA. The nucleic acid can be double-stranded or single-stranded (i.e., a sense or an antisense single strand) and can be complementary to a nucleic acid encoding a polypeptide. Total RNA can be isolated from cells by lysing the cells with sodium dodecyl sulfate (SDS), treating the lysate with proteinase K, and isolating the RNA by extracting with a mixture of 25:24:1 phenol/chloroform/isoamyl alcohol and precipitating with sodium acetate and ethanol. In other methods, cells can be lysed with guanidinium (e.g., 4M guanidium, pH 5.5) and the RNA isolated by cesium chloride purification. Alternatively, total RNA can be extracted with kits such as the Qiagen RNeasy™ kit (Qiagen, Inc., Valencia, Calif.) or the PureScript™ kit (Gentra Systems, Inc., Minneapolis, Minn.).

Routine methods also can be used to extract genomic DNA from a blood or tissue sample, including, for example, phenol extraction. Alternatively, genomic DNA can be extracted with kits such as the QIAamp® Tissue Kit (Qiagen, Chatsworth, Calif.), the Wizard® Genomic DNA purification kit (Promega, Madison, Wis.), the Puregene DNA Isolation System (Gentra Systems, Inc., Minneapolis, Minn.), and the A.S.A.P.™ Genomic DNA isolation kit (Boehringer Mannheim, Indianapolis, Ind.).

To select nucleic acid molecules encoding a TCR or a BCR, or a variable portion of a TCR or BCR, a primer that binds to a region of a TCR or a BCR can be used to produce an extension product in the presence of a polymerase and the appropriate nucleotides. The primer can be designed such that upon extension of the primer, the resulting product encodes a variable portion of a TCR or a BCR. For example, in one embodiment, a primer that binds to a constant region of a TCR or BCR gene is annealed to a sample of total RNA and a complementary DNA (cDNA) is produced using reverse transcriptase and a mixture of deoxynucleotide triphosphates (dNTPs). A second strand can be synthesized using a DNA polymerase, a mixture of dNTPs, and the cDNA as a template. The doubled stranded cDNA product can be purified (e.g., gel-purified) and labeled as discussed below. A complementary RNA (cRNA) product can be produced from the double-stranded cDNA product using RNA polymerase and the appropriate ribonucleotides (NTPs). Alternatively, the second strand can be generated using a reverse primer specific to the region of interest. Primers described above can be designed to include particular promoter sequences to facilitate further manipulations (e.g., in vitro transcription).

In other embodiments, PCR is used to produce nucleic acids encoding a TCR or a BCR, or a variable region of a TCR or BCR. Conventional PCR techniques are disclosed in U.S. Pat. Nos. 4,683,202, 4,683,195, 4,800,159, and 4,965,188. See also, for example, PCR Primer: A Laboratory Manual, Dieffenbach and Dveksler (eds.), Cold Spring Harbor Laboratory Press, 1995) for standard PCR conditions. PCR typically employs two oligonucleotide primers that bind to a selected nucleic acid template (e.g., DNA or RNA, including messenger RNA). Template nucleic acid need not be purified; it can be a minor fraction of a complex mixture, such as microbial nucleic acid contained in mononuclear cells.

Nucleic acid molecules encoding a TCR or BCR, or a variable portion of a TCR or BCR, are labeled, either directly or indirectly, with a detectable label. Typically, the label is incorporated throughout the nucleic acid molecule. For example, the nucleic acids can be labeled during in vitro synthesis of the nucleic acid molecules by incorporating modified dNTPs or NTPs. Alternatively, techniques such as nick-translation or random priming can be used to label a nucleic acid throughout its length.

Nucleic acid molecules can be labeled with an isotope such as ³²P or ³⁵S, a metallic label (e.g., colloidal gold), or can be non-radioactively labeled with a fluorescent nucleotide derivative such as ChromaTide™ (Molecular Probes, Inc., Eugene, Oreg.). In addition, the nucleic acid molecule can be labeled with a fluorophore such as 7-amino-4-methylcoumarin-3-acetic acid (AMCA), Texas Red™ (Molecular Probes, Inc., Eugene, Oreg.), 5-(and-6)-carboxy-X-rhodamine, lissamine rhodamine B, 5-(and-6)-carboxyfluorescein, fluorescein-5-isothiocyanate (FITC), 7-diethylaminocoumarin-3-carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5-(and-6)-carboxamido]hexanoic acid, N-(4,4-difluoro-5,7-dimethyl-4-bora-3a, 4a diaza-3-indacenepropionic acid, eosin-5-isothiocyanate, erythrosin-5-isothiocyanate, phycoerythrin (PE) (B—, R—, or cyanine-), allophycocyanin (APC), peridinin chlorophyll protein (PerCP), Oregon Green™, or Cascade™ blue acetylazide (Molecular Probes, Inc., Eugene, Oreg.). Such molecules allow the hybridization of the two populations of the nucleic acid molecules to be visualized without secondary detection molecules.

Nucleic acid molecules also can be indirectly labeled with biotin or digoxigenin, although secondary detection molecules or further processing then may be required to visualize hybridization of the two populations of nucleic acid molecules. For example, a nucleic acid molecule indirectly labeled with biotin can be detected using avidin or streptavidin conjugated molecules (e.g., avidin or streptavidin conjugated antibodies). Digoxigenin labeled nucleic acids can be detected using anti-digoxigenin antibodies. Typically, the antibodies are conjugated to a reporter molecule such as an enzyme (e.g., alkaline phosphatase or horseradish peroxidase) or a detectable label (e.g., a fluorophore). Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Molecular beacons in conjunction with fluorescence resonance energy transfer (FRET) also can be used as a label. Molecular beacon technology uses a nucleic acid molecule labeled with a first fluorescent moiety and a second fluorescent moiety. The second fluorescent moiety is generally a quencher, and the fluorescent labels are typically located at each end of the nucleic acid. Molecular beacon technology uses a nucleic acid having sequences that permit secondary structure formation (e.g., a hairpin). As a result of secondary structure formation within the nucleic acid, both fluorescent moieties are in spatial proximity when the nucleic acid is in solution. After hybridization to the random nucleic acids, the secondary structure of the nucleic acid is disrupted and the fluorescent moieties become separated from one another such that after excitation with light of a suitable wavelength, the emission of the first fluorescent moiety can be detected.

In some embodiments, the two populations of nucleic acid molecules are not labeled. Hybridization can be assessed using a labeled protein that can distinguish single strands from double strands (e.g., MutS).

Before hybridization to the random nucleic acid molecules, the labeled nucleic acid molecules typically are fragmented into nucleic acid molecules ranging from 20 to 500 nucleotides in length. Fragments of 50 to 200 nucleotides are particularly useful. Fragmenting may not be necessary if the nucleic acids were produced using a reverse primer specific to the region of interest as such nucleic acids will be in an appropriate size range.

Nucleic Acid Molecules From Viruses

To assess viral quasispecies, nucleic acid molecules that encode viral polypeptides are used. A nucleic acid molecule can encode a full-length viral polypeptide or a variable portion thereof. For example, a nucleic acid molecule can encode hemaglutinin of influenza, Env of HIV, gp120 of HIV, or E1 or E2 of hepatitis C, and variable portions of such polypeptides (e.g., hypervariable region 1 of E2 or a portion of hypervariable region 1). Nucleic acid molecules encoding viral polypeptides can be isolated from biological samples and labeled using the techniques described above. In some embodiments, nucleic acid molecules can be obtained from multiple biological samples from the same subject at different time points (e.g., biological samples before and after antibody seroconversion).

Random Nucleic Acid Molecules

Random nucleic acid molecules typically are 10 to 50 nucleotides in length, and preferably, are 20 to 25 nucleotides in length. Random nucleic acids can be of unknown sequence with any one of four nucleotides at each position. Alternatively, random nucleic acid molecules can have known sequences from unrelated genes (i.e., non TCR and non-BCR genes, or non-viral genes).

Populations of random nucleic acid molecules can be produced synthetically. Methods for synthesizing nucleic acid molecules are known in the art. For example, nucleic acid molecules can be assembled by the β cyanoethyl phosphoramidite method. See, for example, “Oligonucleotide Synthesis: A Practical Approach,” ed. M. J. Gait, IRL Press, 1984, WO92/09615; and WO98/08857 for a description of synthesis methods. Automated synthesizer machines can be used to produce random nucleic acid molecules. Such synthesizers are known and are available from a variety of companies including Applied Biosystems and Amersham Pharmacia Biotech.

The random nucleic acid molecules can be attached to a solid substrate. Suitable substrates can be of any shape or form and can be constructed from, for example, glass, silicon, metal, plastic, cellulose, or a composite. For example, a suitable substrate can include a multiwell plate or membrane, a glass slide, a chip, or beads. Suitable beads can have an average diameter of about 2 μm to 15 μm and can be polystyrene, ferromagnetic, or paramagnetic. For example, the beads can have an average diameter of about 4 μm to about 11 μm. Typical average bead diameters are about 4-5 μm, 7-8 μm, and 10-11 μm. Beads are available commercially, for example, from Spherotech Inc., Libertyville, Ill. Nucleic acid molecules can be synthesized in situ, immobilized directly on the substrate, or immobilized via a linker, including by covalent, ionic, or physical linkage. Linkers for immobilizing nucleic acids, including reversible or cleavable linkers, are known in the art. See, for example, U.S. Pat. No. 5,451,683 and WO98/20019. In some embodiments, the nucleic acid molecules are immobilized in discrete locations on the solid substrate (e.g., a chip). See, for example, U.S. Pat. Nos. 5,445,934 and 5,744,305. Such chips are commercially available, e.g., from Affymetrix (Santa Clara, Calif.).

Hybridization

Hybridizations of the two populations of nucleic acid molecules typically are performed under highly stringent conditions. For example, nucleic acid molecules can be hybridized to a chip at 45° C. in 2×MES (2-morpholinoethanesulfonic acid) for 12 to 16 hours then washed twice in 0.5×MES at 65° C. 1×MES contains 1.0M NaCl, 0.1 M MES pH 6.6, and 0.1% Triton X100. Hybridization conditions, including ionic strength of hybridization and wash solutions, temperature of hybridization, length of hybridization, number of washes, and temperature of washes, can be modified to account for unique features of the nucleic acid molecules, including length and overall sequence composition, and the type of solid substrate (e.g., beads or chips).

Methods and systems for imaging samples containing detectable labels are commercially available. For example, a system that includes a scanner, flow cytometer, mass spectrometer, confocal microscope, or real time thermocycler (e.g., with nucleic acid molecules labeled with molecule beacons) can be used to detect hybridization intensity. For example, when the nucleic acid molecules are attached to a bead and a fluorescent label is used, flow cytometry can be used to determine the number and fluorescent intensity of the beads.

Typically, the number of hybridizations with intensity above background (i.e., number of hits) can be summed. Background intensity is determined based on hybridization of a sample with known diversity. In some embodiments, background can be intensity data from non-lymphoid cells. A standard curve in which samples with known numbers of different nucleic acid molecules are hybridized to a random population of nucleic acid molecules can be generated. Diversity of a particular sample can be determined by extrapolating from the standard curve.

In some embodiments, hybridization patterns are analyzed using, for example, the “nearest shrunken centroid” method of analyzing microarrays. See, Tibshirani et al., Proc. Natl. Acad. Sci. USA, 99(10):6567-72 (2002), for a description of nearest shrunken centroid analysis. In general, subsets of binding sites that best characterize each particular population of nucleic acid molecules (e.g., TCR) are identified. A standardized centroid is calculated for each class, where the standardized centroid is defined as the average binding site intensity for each binding site in each class divided by the within-sample standard deviation for that binding site. Nearest centroid classification takes the binding intensity profile of a new sample and compares it to each of these class centroids. The class whole centroid that it is closest to, in squared distance, is the predicted class for that new sample. Nearest shrunken centroid analysis implies the use of a threshold to further shrink the number of binding site subsets for comparison. Determination of the threshold is based on cross-validated misclassification error rate. Nearest shrunken centroid analysis may be particularly useful for tracking individual T cell or B cell clones or clusters of T or B cell clones. See also, Slonim, Nat. Genet., 32 (Suppl.):502-8 (2002) for other data-analysis techniques for microarray data analysis.

Monitoring Disease

Methods of the invention can be used to determine a subject's lymphocyte diversity/repertoire, or to monitor or track diseases in subjects, or to identify particular therapeutic regimens. For example, a subject's lymphocyte diversity can be compared with lymphocyte diversity of a control population. In some embodiments, the control population can be the subject's baseline lymphocyte diversity (e.g., before a particular procedure or before treatment). An alteration in the subject's lymphocyte diversity relative to that of the control population indicates a change in the disease. For example, the method can be used to monitor immune reconstitution following bone marrow transplantation or intensive retroviral therapy. In these settings, a small number of clones might expand by homeostatic proliferation to yield normal lymphocyte numbers, but diversity might be altered.

Loss of diversity has been implicated in various disease states. Thus, changes in diversity can be used to track the progression or remission of disease. Methods of the invention can be used to monitor diseases such as autoimmune disorders (e.g., rheumatoid arthritis, multiple sclerosis, or insulin dependent diabetes mellitus type I), colitis, or lymphoid diseases such as leukemia or lymphoma.

The methods described herein also can be used to track expanded T cell clones or clusters of clones over time based on gene chip hybridization pattern. In particular, expanded T cell clones seen in response to infection, transplantation or homeostatic proliferation may be tracked over time with this method.

In addition, identifying and quantifying viral quasispecies can be used to guide therapeutic choices and make prognostic assessments for subjects. Persistence of some viral infections such as hepatitis and HIV is positively related to the diversity of the virus. For example, in a patient infected with hepatitis C, the quasispecies of the virus can determine whether the infection will resolve or become chronic. In subjects with resolving hepatitis, viral diversity decreases after seroconversion, while in subjects with chronic disease, viral diversity increases after seroconversion.

Articles of Manufacture

Populations of random nucleic acids, solid substrates, or primers can be combined with packaging materials and sold as articles of manufacture or kits (e.g., for determining biologic diversity). Components and methods for producing articles of manufacture are well known. The articles of manufacture may combine one or more components described herein. For example, an article of manufacture can include a solid substrate with random nucleic acid molecules immobilized thereto and primers for producing specific nucleic acid molecules (e.g., nucleic acid molecules encoding a lymphocyte receptor or a portion thereof or a viral nucleic acid molecule). In addition, the articles of manufacture further may include reagents to label nucleic acid molecules, nucleic acids that can serve as positive or negative controls, and/or other useful reagents for determining biologic diversity (e.g., reagents for preparing a standard curve). Nucleic acids that serve as positive or negative controls can be immobilized as a solid substrate. Instructions describing how a population of random nucleic acid molecules can be used to determine biologic diversity also can be included.

The invention will be further described in the following examples, which do not limit the scope of the invention described in the claims.

### EXAMPLES

### Example 1

### Methods and Materials

Isolation of RNA. Spleens harvested from mice were placed in RPMI and pushed through a 70 μm cell strainer. Leukocytes were isolated from the resulting suspension of splenocytes or from peripheral blood using Ficoll-Paque (Amersham Biosciences, Piscatanaway, N.J.) gradient. Total RNA was isolated from the leukocytes using the Qiagen RNeasy kit (Qiagen, Inc., Valencia, Calif.) per the manufacturer's instructions. Isolated RNA was resuspended at a concentration of 2 μg/μl.

Generation of CDR-3 specific CRNA: First strand cDNA was constructed first as follows. In an RNAse-free microcentrifuge tube, 10 μl of total RNA (20 μg) was mixed with 1 μl (100 pmol/μl) of either:

T7+Cβ, which binds to the constant region of the TCR β chain, 5′-GGCCAGTGAATTGTAATACGACTCACTATAGGGAGGCGGCTTGGGTGGAG TCACATTTCTC-3′ (SEQ ID NO: 1) for T cell receptor analysis, or

T7+CJH₄ 5′-GGCCAGTGAATTGTAATACGACTCACTATAGGGAGGCGGGAGGAGACGG TGACTGAGGTTCCTTG-3′ (SEQ ID NO:2) for B cell receptor analysis. The T7+CJH₄ primer binds to the JH₄ region of the receptor.

This mixture was incubated at 70° C. for 10 minutes followed by a quick spin and chill on ice. To this reaction, 4 μl of 5× first strand cDNA buffer (Invitrogen, Inc., Carlsbad, Calif.), 2 μl of 0.1 M DTT, and 1 μl of 10 mM dNTP mix were added and incubated at 37° C. for 2 minutes. Next, 2 μl of SuperScript II Reverse Transcriptase (Invitrogen, Inc.) was added and the total mixture was further incubated at 37° C. for 1 hour.

Following incubation, the first strand product was placed on ice. For second strand synthesis, the following reagents were added to the first strand product: 91 μl of DEPC-treated water, 30 μl of 5× Second Strand Reaction Buffer (Invitrogen, Inc.), 3 μl of 10 mM dNTPs, 1 μl of 10U/μl DNA ligase, 4 μl of 10U/μl DNA polymerase I, and I μl of 2U/μl RNase H. The reaction was incubated at 16° C. for 2 hours in a cooling water bath. Following incubation, 2 μl of 10U T4 DNA polymerase was added and the entire mixture was incubated for an additional 5 minutes at 16° C. Finally, 10 μl of 0.5 M EDTA was added to the mixture. The completed double-stranded cDNA was purified using phase lock gel followed by phenol chloroform extraction. The double-stranded cDNA product was then biotinylated with Enzo, BioArray High Yield RNA Transcript Labeling Kit per the manufacturer's instructions. The in vitro transcription product (cRNA) was purified using RNeasy spin columns (Qiagen, Inc.) per the manufacturer's instructions. The purified product was quantified using spectrophotometric analysis applying the convention that 1 OD at 260 nm equals 40 μg/ml of RNA. cRNA was resuspended at a concentration of 1 μg/μl. cRNA was then fragmented to 50-200 bp sizes by combining with 5 μl of 5× fragmentation buffer (Invitrogen, Inc.) in 15 μl of water. The mixture was incubated at 94° C. for 35 minutes and put on ice following incubation. Equal amounts of cRNA from different samples were hybridized to gene chips as described below.

Application of cRNA to the gene chip. Gene Chips® were purchased from Affymetrix, Inc. (Santa Clara, Calif.) and prepared for hybridization of cRNA. While the ideal gene chip would be synthesized with randomly generated oligonucleotides, it was reasoned that chips containing known but unselected expressed sequence tags from human genes would share less homology with mouse CDR-3 RNA and could be used instead. Accordingly, the human U95B chip was used. As these chips were initially developed for an entirely different purpose, differences in diversity less than one order of magnitude may not be detected. Comparing such differences in diversity can be accomplished with a larger panel of standards; however, there is little evidence such differences matter biologically.

Data Analysis. For each gene chip experiment, raw data representing oligo location and hybridization intensity were obtained. Data were arranged in order of ascending hybridization intensity. The number of oligo locations with intensity above background (i.e., number of hits) was summed. First, the standard curve was generated (from hybridization of samples with known numbers of different oligos). Next, test samples were assessed and based on the number of hits, the diversity was extrapolated from the standard curve.

ELISA for detection of anti-keyhole limpet hemocyanin (KLH) antibodies. Mice were immunized by i.p. injection with 25 μg of KLH (Sigma, St. Louis, Mo.) in complete Freund's adjuvant. A boost of 10 μg of KLH was administered 20 days later. After an additional 2 weeks, the mice were killed and serum and splenocytes were isolated. Purified KLH [3 μg/ml in phosphate buffered saline (PBS), 50 μl/well] was added to wells of 96-well flat bottom microtiter plates (Nunc-Immuno 96 Micro Well-Maxisorp™, Nalge Nunc International, Rochester, N.Y.). ELISA was developed as described by Cascalho et al., Science, 272:1649-1652 (1996). Plates were read using a microplate reader (Power Wave X™; BioTek Instruments, Winooski, Vt.) and analyzed using KC4 Kineticalc software. Samples were analyzed in triplicate ion three independent experiments.

Statistical Analysis

Slope and y intercept of the standard curves were compared between experiments using a single factor analysis of variance for a random effects model. Differences were considered significant at a value of P<0.05. Immune responsiveness was compared between control (C57B1/6) and various mutant mice (QM, JH−/−) using unpaired Student's t-test data. Differences were considered significant at a value of P<0.05. All gene chip experiments were performed twice; representative data are shown.

### Example 2

### Standardization of Analysis of Lymphocyte Receptor Diversity Using Gene Chips

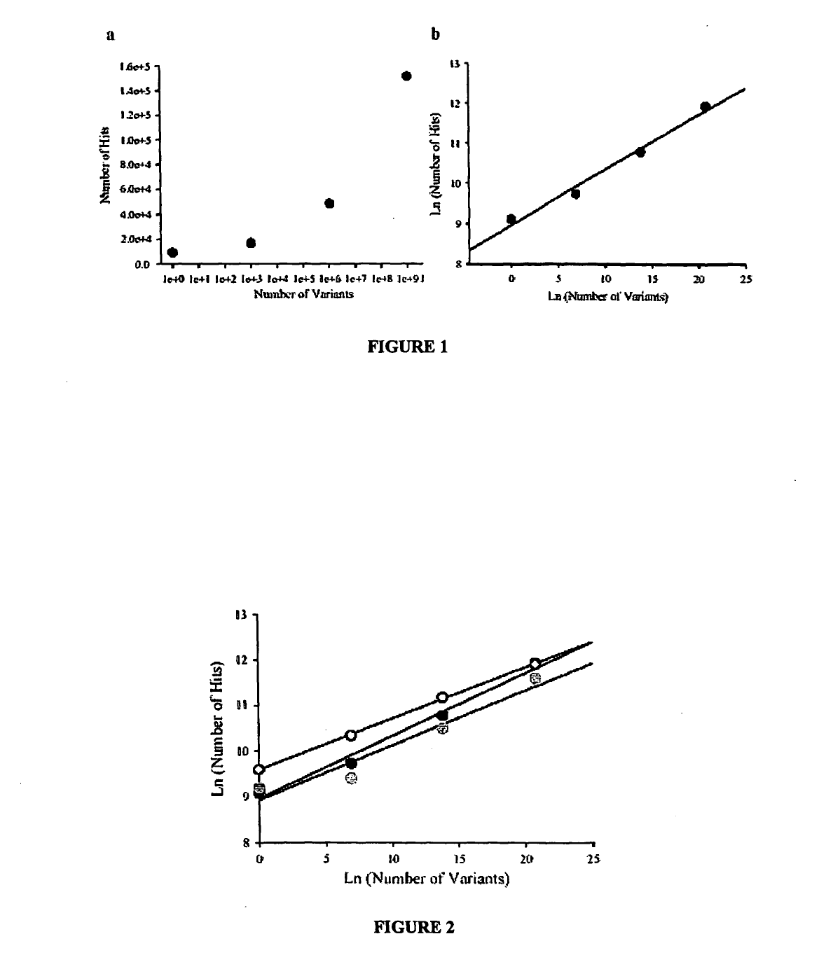

As a first test of the concept, it was assessed whether the diversity of random oligonucleotides could be predicted by the number of sites hybridized on a gene chip. To test this question, an oligonucleotide 18 nucleotides in length (an 18 mer) was designed (similar in sequence to an average T cell receptor CDR3 region) and then random point assignments were inserted at specific locations. For example, to generate a sample with ˜10⁶ different oligonucleotides, an 18 mer was synthesized with 10 sites of random base assignment (4¹⁰=1,048,576). Samples were synthesized with 1, 10³, 10⁶, and 10^{9 *}different oligonucleotides per sample. The oligonucleotides were biotinylated and then 10 μg of each was hybridized to separate gene chips under similar stringency conditions to those for conventional applications (hybridization in 2×MES for 12 to 16 hours at 45° C., washed twice in 0.5×MES at 65° C.). Following hybridization, gene chips were stained with streptavidin phycoerythrin and then scanned using GeneChip software yielding intensity at specific probe locations on the gene chip. Hybridization intensity data were arranged in ascending order. The number of probe locations with intensity above background (i.e., number of hits) was summed and compared to the number of different oligos initially applied to the gene chip (i.e., number of variants). Background was 50. Scans of the gene chips afford rapid inspection of the “hit” profile. As predicted, the number of hybridized sites increased with increasing numbers of different oligonucleotides (FIG. 1A), indicating that a human gene chip could be used to detect random oligonucleotides. Due to the exponential nature of the relationship, the natural logarithm of both variables was taken and plotted (FIG. 1B). The trend (i.e., slope of the standard curve) was highly reproducible, however overall hybridization intensity (i.e., y intercept of the standard curve) varied from experiment to experiment (FIG. 2). This variability requires use of a standard curve for each experiment conducted.

### Example 3

### Analysis of Murine B Cells

To test whether the method of Example 2 could measure variations in lymphocyte diversity, it was applied to the study of B cells in mice. Murine B cells were used for this purpose because diversity can be measured, at least in principle, through analysis of immunoglobulin (Ig) proteins and because of the availability of mutant mice with defined variations in B cell antigen receptor repertoire. Diversity of B cell antigen receptors was compared in wild type (C57B1/6) mice with the in quasi-monoclonal (QM) and monoclonal B-T (MBT) mice. The QM mice were generated by gene-targeted replacement of the endogenous JH elements with a VDJ rearranged region from a (4-hydroxy-3-nitrophenyl) acetyl (NP)-specific hybridoma. Cascalho et al., Science 272:164901652 (1996); and Cascalho et al., J. Immunol. 159:5795-5801 (1997). The kappa light chains in these mice are non-functional and therefore the knock-in heavy chain can only pair with endogenously rearranged lambda chains. All B cells in QM mice start out with the same heavy chain, however secondary rearrangement and hypermutation change the specificity of 20% of B cell receptors in the periphery. Cascalho et al., J. Immunol. supra. Thus, 80% of the peripheral B cells in the QM mice express antibodies containing the same single heavy chain, and the remaining 20% express antibodies expressing diverse heavy chains. The MBT mouse, was produced by breeding recombinase-deficient mice expressing the DO.11 TCR transgene with recombinase deficient mice with a monoclonal B cell compartment that produces antibodies specific for (4-hydroxy-3-nitrophyl) acetyl. Cascalho et al., Science supra; Keshavarzi et al., Scand. J. Immunol., in press. JH −/− mice have a targeted deletion of the JH and of the J kappa gene segments and therefore cannot assemble Ig heavy or kappa light chains. Chen et al., Int. Immunol. 5:647-56 (1993). These animals are B cell deficient although they do have precursor B cells (B220+/CD43+) that assemble lambda light chain genes at a low level.

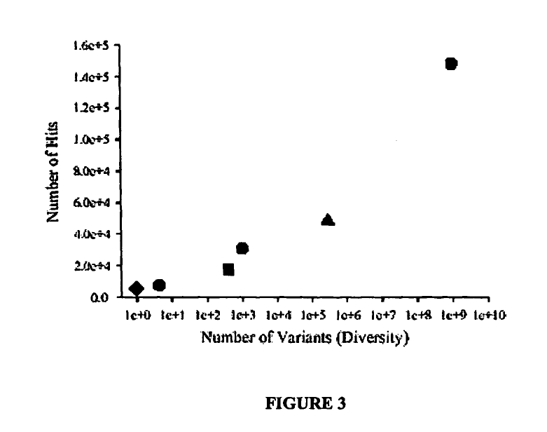

To test the method of measuring lymphocyte diversity, splenocytes were harvested from 3-4 week old JH−/−, MBT, QM and WT mice and mononuclear cells were isolated on Ficoll-paque gradients. Total RNA was isolated from the lymphocytes and first strand cDNA was generated using a primer designed to bind the constant region of the mouse heavy chain J region plus the T7 polymerase promoter. The custom primer promoted amplification of heavy chain-specific RNA only. Equal amounts of the in vitro transcription product (cRNA) from each mouse and standards (-●-) were hybridized to gene chips and then the chips were stained and analyzed as described above in Example 2. The hybridization intensities obtained from the JH −/− mice (which lack B cell receptors) were used to set the background threshold, above which hybridization sites were counted. As the results shown in FIG. 3 indicate, wild type mice expressed more than 10⁵ (2.8×10⁵) different B cell heavy chain clones, QM mice expressed 3.9×10² different heavy chain clones and, as expected, MBT approximately 1 heavy chain clone.

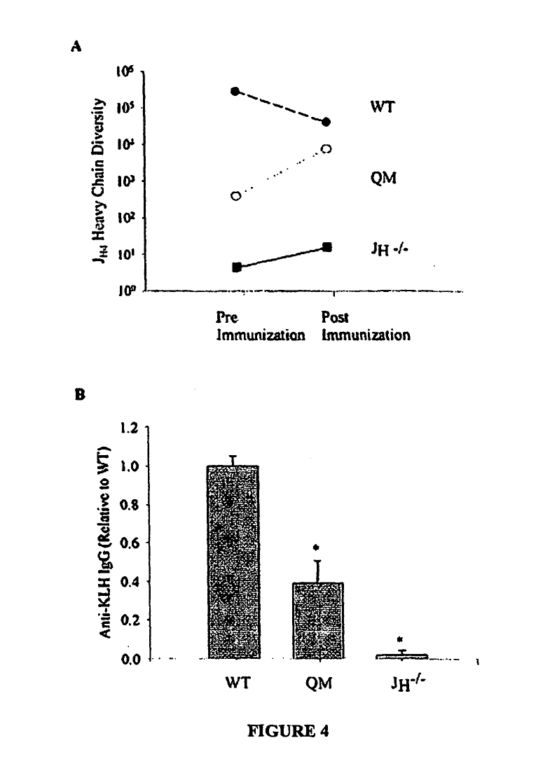

The ability of this system to detect changes in diversity following antigenic challenge with KLH was tested. Following immunization with proteins such as KLH, heavy chain diversity is thought to decrease due to oligoclonal expansion of high affinity clones. In contrast, immunization of QM mice with KLH (an antigen that does not bind with QM antibody) causes diverse non-QM B cells to expand at the expense of the predominant QM B cells, thereby increasing heavy chain diversity. Consistent with these theoretical concepts, JH₄-positive heavy chain diversity in wild-type mice decreased by greater than one order of magnitude, from 2.8×10⁵ to 4.0×10⁴, and diversity in QM mice increased (7.5×10³) following immunization with KLH (FIG. 4A). Immune responsiveness was confirmed by quantitation of anti-KLH Ig in the serum (FIG. 4B).

### Example 4

### Analysis of Human T Cells

To determine if the gene chip method could measure T cell diversity of humans, peripheral blood lymphocytes (PBL) were isolated from five donors (two normal individuals; two thymectomized individuals, and one individual with IBD). Total RNA was isolated from the human peripheral blood lymphocytes or Jurkat cells and first strand cDNA was generated using a primer designed to bind the constant region of the TCR beta chain. The custom primer promoted amplification of TCR-specific RNA only. Equal amounts of the in vitro transcription product (cRNA) from each sample and standards were hybridized to gene chips and then the chips were stained and analyzed as described in Example 2.

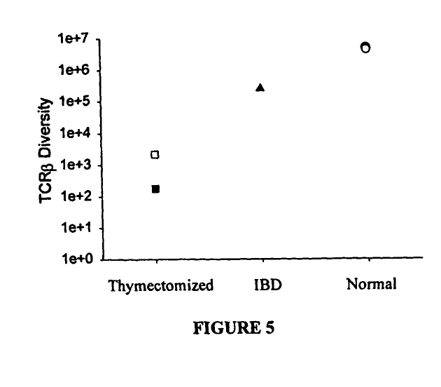

Jurkat cells express only one TCR (Vα1.2Vβ8.1) and so the hybridization intensities of this sample were used to establish the background threshold. The beta chain diversity of the normal individuals was 4.4×10⁶ and 5.1×10⁶, respectively; the beta chain diversity of the thymectomized individuals was 2.2×10³ and 1.8×10², respectively; and the beta chain diversity of the individual with IBD was 2.6×10⁵. See, FIG. 5. The values are consistent with estimates of CDR3 diversity deduced by other means (Wagner et al., Proc. Natl. Acad. Sci. USA 95:14447-52 (1998); Correi-Neves et al., Immunity, 14:21-32(2001)) and taken with estimates of α-chain diversity and pairing (Arstila et al., Science, 286:958-61 (1999)), would place overall T cell diversity near 10⁹. Similar results were obtained for T cell diversity in mice.

### Example 5

### Analysis of Receptor Diversity Using Beads

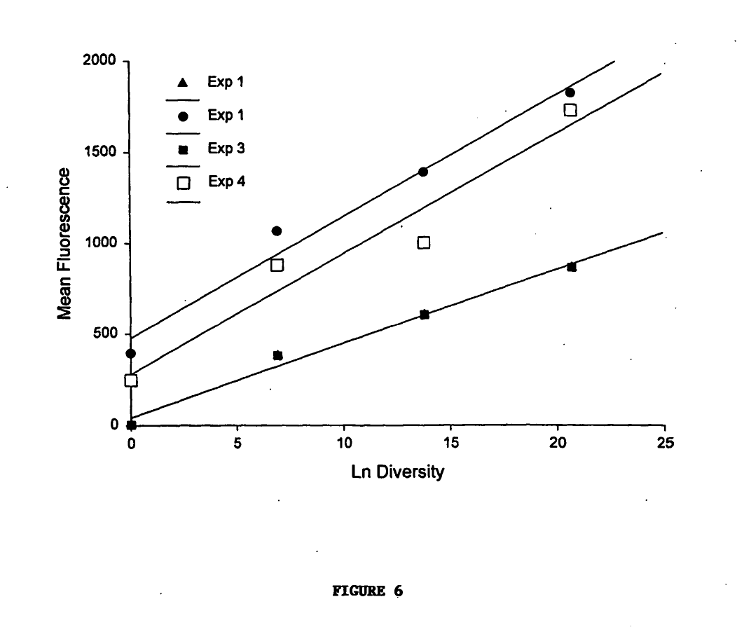

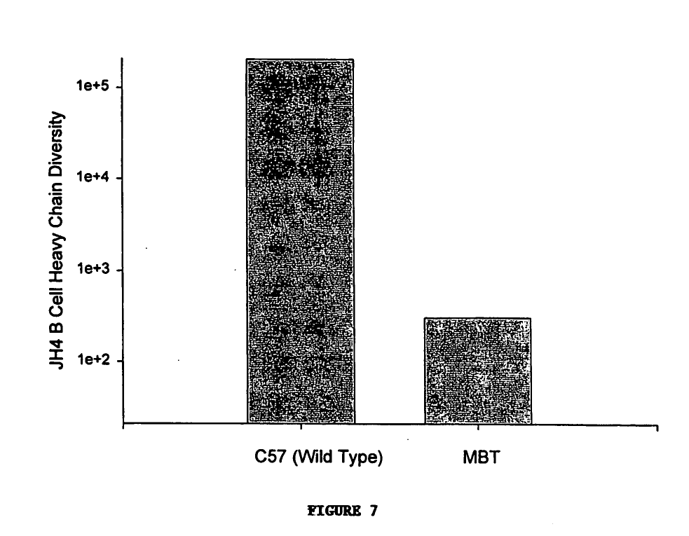

Oligonucleotides can be bound to polymer or glass beads by one of several biochemical binding reactions. It was tested whether random oligonucleotides bound to polymer beads could serve as a substrate for assessing diversity (as shown above using gene chips as a substrate). Random 18mer oligonucleotides were 3′ end-labeled with biotin and allowed to bind to streptavidin-coated polymer beads. Beads with the random oligonucleotides were exposed to one of several samples with known numbers of different oligonucleotides (i.e., diversity) per sample. For example, to generate a sample with ˜10⁶ oligonucleotides, an oligonucleotide was synthesized with 10 sites of random base assignment (4¹⁰=1,048,576). The oligonucleotide samples with known diversity were end-labeled with digoxigenin (DIG). The DIG-labeled samples were hybridized with the oligonucleotide-coated beads for 2 hours. Following hybridization, the beads were washed to remove unbound oligonucleotides and stained with anti-DIG conjugated phycoerythrin and analyzed with a flow cytometer. Mean fluorescence was measured for each sample and is reported as a function of sample diversity (see FIG. 6). As predicted, mean fluorescence increases with increasing sample diversity. The trend and fluorescence intensity varied between the four experiments shown, necessitating use of a standard curve for each experiment conducted. However, fluorescence intensity varies as a linear function of In diversity.

### Example 6

### Analysis of Murine B Cells

To test whether the method of Example 5 could measure variations in lymphocyte diversity, it was applied to the study of B cells in mice. B cells were isolated from wild type (C57B1/6) mice and from MBT mice using magnetic separation techniques. The MBT mouse was produced by breeding recombinase-deficient mice expressing the DO.11 TCR transgene with recombinase-deficient mice with a monoclonal B cell compartment that produces antibodies specific for (4-hydroxy-3-nitrophyl) acetyl. Total RNA was isolated from the B cells and first strand cDNA was generated using a primer end-labeled with DIG and designed to bind the constant region of the mouse heavy chain J_{H4} region. cDNA was treated with RNase and then applied to beads containing random oligonucleotides. Following hybridization, the beads were washed, stained with anti-DIG conjugated phycoerythrin, and analyzed using a flow cytometer as described above. Results from one experiment are shown in FIG. 7. As predicted, WT B cell J_{H4} diversity (1.7×10⁵) is nearly three orders of magnitude greater than that of the B cells of MBT mice (6.4×10²).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining lymphocyte diversity in a subject, said method comprising
a) providing labeled nucleic acid molecules from a population of said subject's lymphocytes, wherein each said labeled nucleic acid molecule encodes a lymphocyte receptor or a portion thereof;
b) hybridizing said labeled nucleic acid molecules or fragments of said labeled nucleic acid molecules with a population of random nucleic acid molecules; and
c) determining lymphocyte diversity of said subject by assessing hybridization of said labeled nucleic acid molecules with said population of random nucleic acid molecules.

2. The method of claim 1, wherein said random nucleic acid molecules within said population are attached to a solid substrate.

3. The method of claim 2, wherein said solid substrate is a multiwell plate or membrane, a glass slide, a chip, or a bead.

4. The method of claim 2, wherein said solid substrate is a bead.

5. The method of claim 4, wherein hybridization is assessed by flow cytometry.

6. The method of claim 2, wherein said solid substrate comprises a plurality of discrete regions, wherein each of said discrete regions comprises a different random nucleic acid molecule.

7. The method of claim 1, wherein said labeled nucleic acid molecules are labeled with a fluorochrome.

8. The method of claim 7, wherein said fluorochrome is fluorescein isothiocyanate (FITC), phycoerythrin (PE), allophycocyanin (APC), or peridinin chlorophyll protein (PerCP).

9. The method of claim 1, wherein said labeled nucleic acid molecules are labeled with biotin.

10. The method of claim 1, wherein said labeled nucleic acid molecules are labeled with an enzyme.

11. The method of claim 1, wherein said population of labeled nucleic acid molecules comprises labeled RNA molecules.

12. The method of claim 1, wherein said population of labeled nucleic acid molecules comprises labeled DNA molecules.

13. The method of claim 1, wherein said population of lymphocytes are T lymphocytes.

14. The method of claim 13, wherein said labeled nucleic acid molecules encode a variable region from a T cell receptor.

15. The method of claim 13, wherein said labeled nucleic acid molecules encode a complementarity determining region (CDR) 3β chain polypeptide.

16. The method of claim 1, wherein said population of lymphocytes are B lymphocytes.

17. The method of claim 16, wherein said labeled nucleic acid molecules encode a variable region from a heavy chain or a light chain.

18. A method for monitoring a disease in a subject, said method comprising
a) providing labeled nucleic acid molecules from a population of said subject's lymphocytes, wherein each said labeled nucleic acid molecule encodes a lymphocyte receptor or a portion thereof;
b) hybridizing said labeled nucleic acid molecules or fragments of said labeled nucleic acid molecules with a population of random nucleic acid molecules;
c) determining lymphocyte diversity of said subject by assessing hybridization of said labeled nucleic acid molecules with said population of random nucleic acid molecules; and
d) comparing said subject's lymphocyte diversity with lymphocyte diversity of a control population, wherein an alteration in said subject's lymphocyte diversity relative to that of said control population indicates a change in said disease.

19. The method of claim 18, wherein an increase in said subject's lymphocyte diversity indicates a positive change in said disease.

20. The method of claim 18, wherein a decrease in said subject's lymphocyte diversity indicates a negative change in said disease.

21. The method of claim 18, wherein said disease is an autoimmune disorder.

22. The method of claim 21, wherein said autoimmune disorder is rheumatoid arthritis or multiple sclerosis.

23. The method of claim 21, wherein said disease is colitis.

24. The method of claim 18, wherein said disease is a lymphoid disease.

25. The method of claim 24, wherein said disease is leukemia.

26. The method of claim 24, wherein said disease is lymphoma.

27. The method of claim 18, wherein said random nucleic acid molecules within said population are attached to a solid substrate.

28. The method of claim 27, wherein said solid substrate is a multiwell plate or membrane, a glass slide, a chip, or a bead.

29. The method of claim 27, wherein said solid substrate is a bead.

30. The method of claim 29, wherein hybridization is assessed by flow cytometry.

31. The method of claim 27, wherein said solid substrate comprises a plurality of discrete regions, wherein each of said discrete regions comprises a different random nucleic acid molecule.

32. The method of claim 18, wherein said labeled nucleic acid molecules are labeled with a fluorochrome, biotin, or an enzyme.

33. The method of claim 32, wherein said fluorochrome is FITC, PE, APC, or PerCP.

34. The method of claim 18, wherein said population of labeled nucleic acid molecules comprises labeled RNA molecules.

35. The method of claim 18, wherein said population of labeled nucleic acid molecules comprises labeled DNA molecules.

36. A method for determining viral diversity in a subject, said method comprising
a) providing labeled nucleic acid molecules from a biological sample of said subject, wherein said labeled nucleic acid molecules encode a viral polypeptide;
b) hybridizing said labeled nucleic acid molecules or fragments of said labeled nucleic acid molecules with a population of random nucleic acid molecules; and
c) determining viral diversity of said subject by assessing hybridization of said labeled nucleic acid molecules with said population of random nucleic acid molecules.

37. The method of claim 36, wherein said random nucleic acid molecules within said population are attached to a solid substrate.

38. The method of claim 37, wherein said solid substrate is a multiwell plate or membrane, a glass slide, a chip, or a bead.

39. The method of claim 37, wherein said solid substrate is a bead.

40. The method of claim 39, wherein hybridization is assessed by flow cytometry.

41. The method of claim 37, wherein said solid substrate comprises a plurality of discrete regions, wherein each of said discrete regions comprises a different random nucleic acid molecule.

42. The method of claim 38, wherein said labeled nucleic acid molecules are labeled with a fluorochrome, biotin, or an enzyme.

43. The method of claim 42, wherein said fluorochrome is FITC, PE, APC, or PerCP.

44. The method of claim 36, wherein said population of labeled nucleic acid molecules comprises labeled RNA molecules.

45. The method of claim 36, wherein said population of labeled nucleic acid molecules comprises labeled DNA molecules.

46. The method of claim 36, wherein said viral polypeptide is hemaglutinin, Env, gp120, E1, or E2, or a variable portion thereof.

47. An article of manufacture comprising a) a solid substrate comprising random nucleic acid molecules immobilized thereto; and b) a primer for producing nucleic acid molecules encoding a lymphocyte receptor or a fragment thereof or a primer for producing nucleic acid molecules encoding a viral polypeptide.

48. The article of manufacture of claim 47, wherein said solid substrate is a multiwell plate or membrane, a glass slide, a chip, or a bead.

49. The article of manufacture of claim 47, wherein said solid substrate is a bead.

50. The article of manufacture of claim 47, wherein said solid substrate is a chip.
